SEQUENCE LISTING (8 sequences)

SEQ ID NO: 1 - DNA, 15 bp, Artificial Sequence
  An exemplary oligonucleotide of the present invention
  misc_structure (8): n is fluorophore-labeled 2′-deoxyuridine
tggactcnctcaatg

SEQ ID NO: 2 - DNA, 15 bp, Artificial Sequence
  Oligonucleotide having single base noncomplementary to SEQ NO 1
cattgagtgagtcca

SEQ ID NO: 3 - DNA, 15 bp, Artificial Sequence
  Oligonucleotide having single base noncomplementary to SEQ NO 1
cattgagggagtcca

SEQ ID NO: 4 - DNA, 15 bp, Artificial Sequence
  Oligonucleotide having single base noncomplementary to SEQ NO 1
cattgagcgagtcca

SEQ ID NO: 5 - DNA, 15 bp, Artificial Sequence
  Oligonucleotide having complementary to SEQ NO 1
cattgagagagtcca

SEQ ID NO: 6 - DNA, 19 bp, Artificial Sequence
  An exemplary oligonucleotide of the present invention
  modified_base (10): n is fluorophore-labeled 2′-deoxyuridine
ttctgactcnctttcagaa

SEQ ID NO: 7 - DNA, 19 bp, Artificial Sequence
  Oligonucleotide having complementary to SEQ NO 6
ttctgaaagagagtcagaa

SEQ ID NO: 8 - DNA, 19 bp, Artificial Sequence
  Oligonucleotide having single base noncomplementary to SEQ NO 6
ttctgaaagcgagtcagaa

(19) United States
(12) Patent Application Publication
(10) Pub. No.: US 2008/0032413 A1
(43) Pub. Date: Feb. 7, 2008

(54) Oligonucleotide For Detecting Target Dna Or Rna

(76) Inventors: Byeang-Hyean Kim, Pohang-si (KR); Gil-Tae Hwang, Pohang-si (KR); Young-Jun Seo, Pohang-si (KR)

Correspondence Address: SUGHRUE MION, PLLC, 2100 PENNSYLVANIA AVENUE, N.W., SUITE 800, WASHINGTON, DC, 20037, US

(21) Appl. No.: 11/578,058

(22) PCT Filed: Mar. 15, 2005
(86) PCT No.: PCT/KR05/00729

§ 371 Date: Oct. 12, 2006

Related U.S. Application Data
(60) Provisional application No. 60/561,146, filed on Apr. 12, 2004.

(51) Int. Cl. G01N 21/64; C07H 21/04

(52) U.S. Cl. 436/172; 536/24.3

(57) ABSTRACT

An oligonucleotide that can be used for detecting the presence of a target DNA or RNA in a sample, a method for detecting a target DNA or RNA by using the oligonucleotide, and a kit comprising the oligonucleotide are provided. The oligonucleotide comprises a nucleoside labeled with a fluorophore and at least one neighboring nucleoside positioned next to the fluorophore-labeled nucleoside, the nucleobase of the neighboring nucleoside being thymine or cytosine.

### FIELD OF THE INVENTION

The present invention relates to an oligonucleotide for detecting a target DNA or RNA, which comprises a nucleoside labeled with a fluorophore and at least one specific nucleoside positioned next to the fluorophore-labeled nucleoside.

### BACKGROUND OF THE INVENTION

Sequence-selective DNA detection is a powerful tool for monitoring many biological processes in the biotechnology field. A novel class of oligonucleotide probes, commonly referred to as molecular beacons, have been developed to facilitate the detection of specific nucleic acid target sequences (see Piatek et al., 1998, Nature Biotechnol. 16:359-363; and Tyagi and Kramer, 1996, Nature Biotechnol. 14:303-308). A molecular beacon is a nucleic acid sequence that has a fluorophore and a quencher at the 5′ and 3′ ends, respectively. A molecular beacon forms a stem-loop structure, and when it receives a light that can excite the fluorophore, the fluorescence emitted from the fluorophore is absorbed by the quencher.

A molecular beacon is designed to have a base sequence complementary to that of a target DNA or RNA of interest. When the molecular beacon meets a target sequence which is complementary to that of the molecular beacon, hybridization between the sequences occurs to form a double helix, and the torsional force generated as the result causes the stem region of the molecular beacon to unwind. As a consequence, the fluorophore is pulled apart from the quencher, thereby negating the role of the quencher.

However, the traditional molecular beacon has the following disadvantages: First, it is capable of detecting only a target DNA or RNA having a sequence which is fully complementary to that of the molecular beacon; second, as its ends are occupied by a fluorophore and a quencher, there is no room to attach any useful functional group which can be used, e.g., for fixing the molecular beacon on a substrate; and third, a complicated and costly process must be employed to attach a quencher.

Therefore, the present inventors have endeavored to develop a new oligonucleotide probe system, which is devoid of the above problems.

### SUMMARY OF THE INVENTION

Accordingly, it is an object of the present invention to provide an oligonucleotide for detecting a target DNA or RNA, which comprises (i) a nucleoside labeled with a fluorophore and (ii) at least one nucleoside having thymine or cytosine nucleobase, which is positioned next to the fluorophore-labeled nucloside.

It is another object of the present invention to provide a method for detecting the presence of a target DNA or RNA by way of using said oligonucleotide.

It is a further object of the present invention to provide a kit for detecting a target DNA or RNA, which comprises said oligonucleotide.

### BRIEF DESCRIPTION OF THE DRAWINGS

The above and other objects and features of the present invention will become apparent from the following description of the invention, when taken in conjunction with the accompanying drawings, which respectively show:

FIG. 1: a schematic diagram for preparing 2′-deoxyuridine labeled with a fluorophore;

FIG. 2: an exemplary oligonucleotide of the present invention (SEQ ID NO: 1);

FIG. 3: the fluorescence spectra observed for the fully matched nucleotides (SEQ ID NOs: 1 and 5) and the single-base-mismatched nucleotides (SEQ ID NOs: 1 and 2, 1 and 3, and 1 and 4);

FIG. 4: the stem-loop structure of the SEQ ID NO: 6; and

FIG. 5: the fluorescence spectra observed for the fully matched nucleotide (SEQ ID NOs: 6 and 7) and the single-base-mismatched nucleotides (SEQ ID NOs: 6 and 8).

### DETAILED DESCRIPTION OF THE INVENTION

The present invention relates to an oligonucleotide for detecting a target DNA or RNA, which comprises (i) a nucleoside labeled with a fluorophore and (ii) at least one nucleoside having thymine or cytosine nucleobase, which is positioned next to the fluorophore-labeled nucleoside.

The oligonucleotide of the present invention is characterized in that it contains a fluorophore without a quencher.

In the present invention, a nucleoside labeled with a fluorophore, such as 2′-deoxyuridine labeled with a fluorophore, may be prepared by a method known in the relevant art. The fluorophore may be selected from the group consisting of fluorene, pyrene, fluorescein, rhodamine and coumarin; preferably, fluorene.

The oligonucleotide of the present invention is designed to contain a nucleoside labeled with a fluorophore and at least one nucleoside having thymine or cytosine nucleobase, which is positioned next to the fluorophore-labeled nucleoside. For preparing the oligonucleotide of the present invention, any one of the methods known in the art for synthesizing an oligonucleotide may be employed. Preferably, an automated DNA synthesizer is employed.

The fluorophore may be located at any position within the oligonucleotide, but the fluorophore is preferably positioned at the center of the oligonucleotide.

The oligonucleotide of the present invention is also characterized in that the thymine or cytosine-based nucleoside located next to the fluorophore-labeled nucleoside plays an important role in quenching the fluorescence emitted from the fluorophore, which makes it unnecessary to employ a quencher.

When the oligonucleotide of the present invention hybridizes with a target DNA or RNA having a fully matched base sequence, the fluorescence intensity dramatically increases over that of a free oligonucleotide. When the sample examined contains a DNA or RNA having only one mismatch with the sequence of the oligonucleotide of the present invention, on the other hand, the fluorescence intensity markedly decreases as compared with that of a free oligonucleotide. Accordingly, the oligonucleotide of the present invention can be advantageously used for detecting the presence of a target DNA or RNA having a fully matched or single-base-mismatched sequence in a sample.

An exemplary oligonucleotide of the present invention has any one of the base sequences of SEQ ID NOs: 1 and 6 (see FIGS. 2 and 4).

The oligonucleotide of the present invention may form a stem-loop structure like the traditional molecular beacons, but it is not limited to a class of oligonucleotides that form stem-loop structures.

The oligonucleotide of the present invention can be used for detecting the presence of a target DNA or RNA having a base sequence completely matched or single-base-mismatched with that of the oligonucleotide. Specifically, the oligonucleotide of the present invention is allowed to hybridize with DNAs or RNAs in a sample, and then the fluorescence intensity is measured to see whether the fluorescence intensity increases or decreases as compared with that of a free oligonucleotide. When the sample contains a DNA or RNA having a base sequence complementary to the inventive oligonucloetide, the fluorescence intensity increase by a factor of two (2) or more over that of a free form of the oligonucleotide, whereas when a DNA or RNA having a single-base-mismatched base sequence is present in the sample, the fluorescence intensity decreases by a magnitude of 0.1 to 0.3 fold as compared with that of a free oligonucleotide. Accordingly, the oligonucleotide of the present invention is capable of detecting a DNA or RNA having a completely matched or single-base mismatched sequence. Therefore, the oligonucleotide of the present invention can be used as an efficient fluorescence ON/OFF system for detecting a DNA or RNA having a fully matched or single-base mismatched base sequence.

As the oligonucleotide of the present invention does not contain any quencher at its end, its preparation process is simple, and the free end is available for the introduction of any functional group that can be advantageously exploited for extended application.

The present invention also provides a method for detecting the presence of a target DNA or RNA in a sample, which comprises (i) allowing the oligonucleotides of the present invention to react with the sample to let any possible hybridization occur; (ii) measuring the intensity of the fluorescence emitted from the hybridization mixture; and (iii) determining whether the target DNA or RNA is present in the sample.

The present invention further provides a kit for detecting a target DNA or RNA, which comprises the oligonucleotide of the present invention. The kit may further comprise a conventional buffer, additive, etc. known in the relevant art used for hybridization.

The following Examples are intended to further illustrate the present invention without limiting its scope.

### EXAMPLE

¹H, ¹³C and ³¹P NMR spectra were obtained using a Bruker NMR spectrometer (Aspect 300 MHz), FAB mass spectra and a JEOL four sector tandem mass spectrometer (JMS-HX/HX110A). Further, percentages given below for solid in solid mixture, liquid in liquid, and solid in liquid mixture are on wt/wt, vol/vol and wt/vol base, respectively. All reactions were carried out at room temperature, unless specifically indicated otherwise.

### Example 1

### Preparation of 2′-deoxyuridine labeled with fluorophore

(1-1) Preparation of 5′-O-[Bis(4-methoxyphenyl)phenylmethyl]-2′-deoxy-5-(2-ethynylfluorenyl)uridine

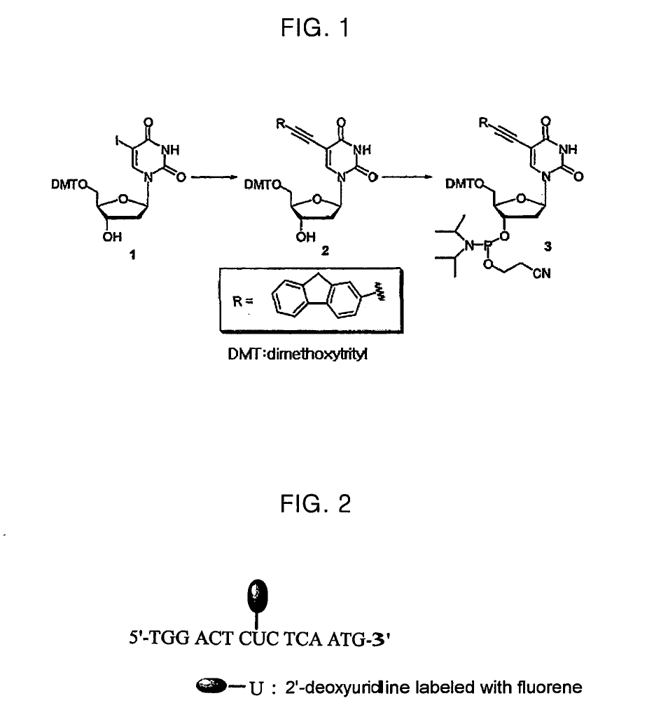

(PPh₃)₂PdCl₂ (53 mg, 0.076 mmol) and CuI (14 mg, 0.074 mmol) were added to a solution obtained by dissolving 5-iodo-5′-dimethoxytrityl-2′-deoxyuridine (497 mg, 0.757 mmol) (See compound 1 of FIG. 1) and 2-ethynylfluorene (231 mg, 1.21 mmol) in Et₃N (4 mL) and THF (12 mL). Argon was bubbled through the mixture for 2 min, and the mixture was subjected to ten (10) pump/purge cycles. Then, the mixture was stirred at 45 to 50° C. for 2 h, and the solvent was evaporated under a reduced pressure. The resultant residue was subjected to column chromatography (Merck 60 silica gel, 230-400 mesh) using the eluting solution of hexane/EtOAc(1:5), to obtain the title compound (434 mg, 80%), which was recrystallized from CHCl₃/MeOH (1:1) (See compound 2 of FIG. 1)

M.p. 160-161° C.

[α]¹⁴_{D}=+40° (c=1.05, CHCl₃).

IR (film): ν 3425, 3185, 3013, 2932, 2836, 2261, 1700, 1608, 1508, 1453, 1251, 1177, 1094, 1034, 829, 767, 701, 668 cm^{31 1}.

¹H NMR (300 MHz, CDCl₃): δ10.26 (br s, 1H; NH), 8.32 (s, 1H; H-6), 7.73 (d, J=7.4 Hz, 1H: fluorene-H), 7.58-7.51 (m, 4H; fluorene-H), 7.43-7.26 (m, 2H+6H; fluorene-H and DMT-H), 7.15 (d, J=7.5 Hz, 2H; DMT-H), 7.04 (br s, 1H; DMT-H), 6.83 and 6.81 (2d, J=8.5 Hz, 4H; DMT-H), 6.48 (t, J=5.9 Hz, 1H; H-1′), 4.63 (br s, 1H; H-3′), 4.26 (br s, 1H; H-4′), 3.99 (br s, 1H; OH), 3.71 (s, 2H; ArCH₂), 3.64 and 3.63 (2s, 6H; OCH₃), 3.50 (br d, J=9.2 Hz, 1H; H-5′), 3.33 (br d, J=8.1 Hz, 1H; H-5′), 2.68 (br s, 1H; H-2′), 2.39 (br s, 1H; H-2′).

¹³C NMR (75 MHz, CDCl₃): δ 162.0, 158.3, 149.6, 144.4, 143.4, 142.5, 142.0, 141.5, 140.9, 135.5, 135.4, 130.2, 129.9, 129.8, 128.1, 127.9, 127.8, 126.9, 126.7, 124.9, 120.3, 120.0, 119.2, 113.2, 100.7, 94.5, 86.8, 85.9, 79.9, 77.2, 72.3, 63.5, 55.0, 41.5, 36.5.

HRMS-FAB (m/z): [M+Na]⁺ calcd for C₄₅H₃₈N₂O₇Na, 741.2578; found, 741.2577.

### (1-2) Preparation of 5′-O-[Bis(4-methoxyphenyl)phenylmethyl]-2′-deoxy-5-(2-ethynylfluorenyl)-3′-[2-cyanoethylbis(1-methylethyl)phosphoramidyl]uridine

2-cyanoethyldiisopropyl chlorophosphoramidite (120 μL, 0.537 mmol) was added dropwise to a solution obtained by dissolving the compound prepared in Example (1-1) (301 mg, 0.419 mmol) and 4-methylmorpholine (140 μL, 1.27 mmol) in CH₂-Cl₂ (12 mL) at room temperature. After the reaction reached completion (about 30 min), the mixture was concentrated under a reduced pressure and then purified by column chromatography (Merck 60 silica gel, 230-400 mesh) using hexane/EtOAc (1:1), to obtain the title compound (285 mg, 74%) (See compound 3 of FIG. 1).

M.p. 98-100° C.

[α]¹⁴_{D}=+35° (c=0.995, CHCl₃).

IR (film): ε 3186, 3016, 2967, 2837, 2254, 1699, 1608, 1581, 1508, 1455, 1364, 1304, 1251, 1178, 1155, 1035, 880, 830, 771, 667 cm⁻¹.

¹H NMR (300 MHz, CDCl₃): δ8.33 and 8.28 (2s, 1H; NH), 7.74 and 7.71 (2s, 1H; H-6), 7.57-7.48 (m, 4H; fluorene-HE), 7.40-7.26 (m, 3H+6H; fluorene-H+DMT-H), 7.17-7.05 (m, 2H; DMT-H), 6.99 and 6.95 (2s, 1H; DMT-H), 6.80 (d, J=8.7 Hz, 4H; DMT-H), 6.39 (dd, J=12.7, 5.6 Hz, 1H; H-1′), 4.63 (br s, 1H; H-3′), 4.26 and 4.21 (2br s, 1H; H-4′), 3.88-3.75 (m, 1H; PCH₂), 3.71 and 3.70 (2s, 2H; ArCH₂), 3.67 and 3.65 (2s, 6H; OCH₃), 3.64-3.50 (m, 4H; NCH, PCH₂, H-5′), 3.32-3.28 (m, 1H; H-5′), 2.66-2.58 (m, 2H; CH₂CN, H-2′), 2.46-2.35 (m, 2H; CH₂CN, H-2′), 1.18 and 1.07 (2d, J=6.7 Hz, 12H; NCHCH₃).

¹³C NMR (75 MHz, CDCl₃): δ 161.4, 158.5, 149.2, 1443, 144.3, 143.5, 142.5, 141.8, 141.6, 141.0, 135.4, 130.3, 129.9, 128.2, 128.0, 127.0, 126.8, 125.0, 120.4, 120.1, 119.2, 117.6, 117.4, 113.2, 100.8, 94.5, 87.0, 86.2, 85.7, 79.8, 63.2, 63.0, 58.2, 58.0, 55.1, 43.3, 43.2, 43.1, 43.0, 40.8, 36.5, 24.6, 24.5, 20.4, 20.3, 20.2, 20.1, 19.2.

³¹P NMR (121 MHz, CDCl₃): δ 151.6, 151.1.

HRMS-FAB (m/z): [M+Na]⁺ calcd for C₅₄H₅₅N₄O₈PNa, 941.3658; found, 941.3654.

### Example 2

### Synthesis of the oligonucleotides of the Present Invention

The compound obtained in Example (1-2) was introduced as a building block to prepare the fluorescent oligonucleotides of SEQ ID NOs: 1 and 6 on a Controlled Pore Glass 9CPG solid support by the phosphoramidite approach using an automated DNA synthesizer (PerSeptive Biosystems 8909 Expedite™ Nucleic Acid Synthesis System). The oligonucleotides prepared were characterized by MALDI-TOF mass spectrometry as follow:

The oligonucleotide of SEQ ID NO: 1, calcd m/z 4717, found 4723; and the oligonucleotide of SEQ ID NO: 6, calcd m/z 5903, found 5903.

Further, the oligonucleotides of SEQ ID NOs: 2 to 5, 7 and 8, candidate target DNAs, were prepared using the same automated DNA synthesizer.

TABLE 1

Base sequence (5′→3′)

| | |
|---|---|
| Oligonucleotide of | TGG ACT CN* C TCA ATG |
| SEQ ID NO: 1 | |
| Oligonucleotide of | CAT TGA GTG AGT CCA |
| SEQ ID NO: 2 | |
| Oligonucleotide of | CAT TGA GGG AGT CCA |
| SEQ ID NO: 3 | |
| Oligonucleotide of | CAT TGA GCG AGT CCA |
| SEQ ID NO: 4 | |
| Oligonucleotide of | CAT TGA GAG AGT CCA |
| SEQ ID NO: 5 | |
| Oligonucleotide of | TTC TGA CTC N* CTT TCA GAA |
| SEQ ID NO: 6 | |
| Oligonucleotide of | TTC TGA AAG A GAG TCA GAA |
| SEQ ID NO: 7 | |
| Oligonucleotide of | TTC TGA AAG C GAG TCA GAA |
| SEQ ID NO: 8 | |

N* is 2′-deoxyuridiue labeled with fluorophore

As seen in Table 1, the oligonucleotides of SEQ ID NOs: 5 and 7 have base sequences complementary to SEQ ID NOs: 1 and 6, respectively. Oligonucleotides of SEQ ID NOs: 2 to 4, and that of SEQ ID NO: 8, on the other hand, have base sequences having one-base-mismatch with SEQ ID NOs: 1 and 6, respectively.

The synthesized oligonucleotides were cleaved from the solid support by treatment with 30% aqueous NH₄OH (1.0 mL) for 10 h at 55° C. The crude products obtained from the automated oligonucleotide synthesis were lyophilized and diluted with distilled water (1 mL). The oligonucleotides were purified by HPLC (Merck LichoSPHER® 100 RP-18 endcapped column, 10×250 mm, 5 μm). The HPLC mobile phase was held isocratically for 10 min with 5% acetonitrile/0.1 M triethylammonium acetate (TEAA) (pH 7.0) at a flow rate of 2.5 mL/min. Then, the gradient was linearly increased over 10 min from 5% acetonitrile/0.1 M TEAA to 50% acetonitrile/0.1 M TEAA at the same flow rate. The fractions containing the purified oligenucleotide were pooled and lyophilized. 80% aqueous AcOH was added to the oligonucleotide. After 30 min at ambient temperature, the solvent was evaporated under a reduced pressure. The residue was diluted with distilled water (1 mL), and then purified by HPLC under the same condition as described above. For characterization, matrix-assisted laser-desorption-ionization time-of-flight (MALDI-TOF) mass spectrometric data of the oligonucleotides were collected with a Voyager RP (PerSeptive Biosystems, Framingham, Mass., USA) time-of-flight (TOF) dual-stage reflector mass spectrometer. The instrument used a nitrogen laser at 337 nm to desorb/ionize the samples. The accelerating voltage was 20 kV and the flight path was 1.1 m.

### Example 3

### The measurement of Fluorescence Intensity

The fluorescent oligonucleotides of the present invention (SEQ ID NOs: 1 and 6) were examined in terms of whether they can be used to detect a target having completely matched or single-base mismatched base sequence, as follows:

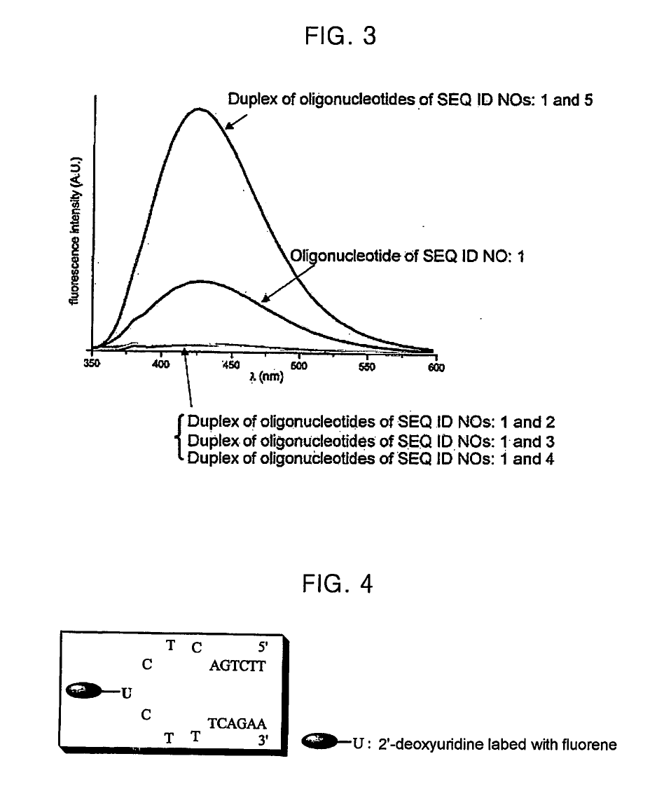

The oligonucleotide of SEQ ID NO: 1 was hybridized with each of the oligonucleotides of SEQ ID NOs: 2 to 5, in a molar ratio of 1:1 in a buffer (100 mM NaCl, 20 mM MgCl₂ and 10 mM Tris-HCl buffer (pH 7.2)), and its steady-state fluorescence (FL) spectrum was taken with a MD-5020 PTI model microscope photometer using a bandwidth of 15 nm and 0.5×2 cm quartz cuvettes with a light pass of 1 cm. The cell holder was thermostated with circulating water controlled by a PolyScience digital temperature controller 9110. The fluorescence measurement was carried out in the same buffer as used in the hybridization. Fluorescence emission spectra are shown in FIG. 3. The fluorescence intensities measured at λₘₐₓ of 425 nm are listed in Table 2.

TABLE 2

| | Fluorescence intensity |
|---|---|
| | relative to that oligonucleotide |
| | of SEQ ID NO: 1 (%) |
| Duplex of oligonucleotides of SEQ ID | 12 |
| NOs: 1 and 2 | |
| Duplex of oligonucleotides of SEQ ID | 12 |
| NOs: 1 and 3 | |
| Duplex of oligonucleotides of SEQ ID | 24 |
| NOs: 1 and 4 | |
| Duplex of oligonucleotides of SEQ ID | 340 |
| NOs: 1 and 5 | |

Accordingly, in case of duplexes wherein all base parings between the oligonucleotides were completely matched (duplexes of oligonucleotides of SEQ ID NOs: 1 and 5), the fluorescence intensity markedly increased. On the other hand, in case of duplexes wherein one base paring was mismatched (duplexes of the oligonucleotides of SEQ ID NOs: 1 and 2, 1 and 3, and 1 and 4), the fluorescence intensity dramatically decreased.

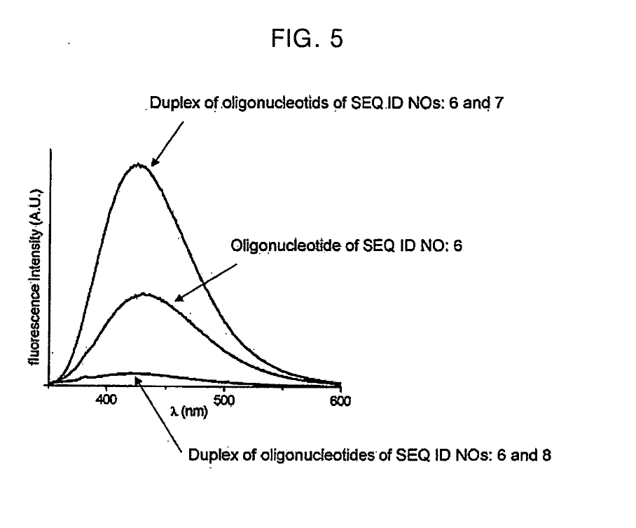

Further, the above experiment was repeated for the fluorescent oligonucleotide of SEQ ID NO: 6 except for using oligonucleotides of SEQ ID NOs: 7 and 8, instead of those of SEQ ID NOs: 2 to 5. The observed fluorescence emission spectra are shown in FIG. 5, and the fluorescence intensities measured at 425 nm are listed in Table 3.

TABLE 3

| | Fluorescence intensity relative to that |
|---|---|
| | oligonucleotide of SEQ ID NO: 6 (%) |
| Duplex of oligonucleotides of | 220 |
| SEQ ID NOs: 6 and 7 | |
| Duplex of oligonucleotides of | 15 |
| SEQ ID NOs: 6 and 8 | |

Thus, when all base parings between the oligonucleotides were completely matched (duplexes of the oligonucleotides of SEQ ID NOs: 6 and 7), the fluorescence intensity increased by a large factor, whereas in case of duplexes wherein one base paring between the oligonucleotides was mismatched (duplexes of the oligonucleotides of SEQ ID NOs: 6 and 8), the fluorescence intensity markedly decreased.

Thus, it was confirmed that the oligonucleotide of the present invention can be advantageously used for detecting a target DNA or RNA having either a fully matched or single-base-mismatched base sequence, by way of measuring the change in the fluorescence intensity.

While the invention has been described with respect to the above specific embodiments, it should be recognized that various modifications and changes may be made to the invention by those skilled in the art which also fall within the scope of the invention as defined by the appended claims.

What is claimed is:

1. An oligonucleotide for detecting a target DNA or RNA, which comprises (i) a nucleoside labeled with a fluorophore and (ii) at least one nucleoside having thymine or cytosine nucleobase, which is positioned next to the fluorophore-labeled nucleoside.

2. The oligonucleotide of claim 1, wherein the nucleoside of the fluorophore-labeled nucleoside is 2′-deoxyuridine.

3. The oligonucleotide of claim 1, wherein the fluorophore is positioned at the center of oligonucleotide.

4. The oligonucleotide of claim 1, wherein the fluorophore is selected from the group consisting of fluorene, pyrene, fluorescein, rhodamine and coumarin.

5. The oligonucleotide of claim 4, wherein the fluorophore is fluorene.

6. The oligonucleotide of any one of claims 1 to 5, which has the base sequence of SEQ ID NO: 1.

7. The oligonucleotide of any one of claims 1 to 5, which forms a stem-loop structure.

8. The oligonucleotide of claim 7, which has the base sequence of SEQ ID NO: 6.

9. A method for detecting the presence of a target DNA or RNA in a sample, which comprises:
i) allowing the oligonucleotide of any one of claims 1 to 5 to react with the sample to let any possible hybridization occur;
ii) measuring the intensity of the fluorescence emitted from the hybridization mixture; and
iii) determining whether the target DNA or RNA is present in the sample.

10. A kit for detecting a target DNA or RNA, which comprises the oligonucleotide of any one of claims 1 to 5.
